(19) United States
(12) Patent Application Publication
(10) Pub. No.: US 2014/0180070 A1
(43) Pub. Date: Jun. 26, 2014

(54) INTRALUMINAL IMAGING SYSTEM

(71) Applicant: VOLCANO CORPORATION, San Diego, CA (US)

(72) Inventors: Bret Millett, Folsom, CA (US); Paul Hoseit, El Dorado Hills, CA (US)

(73) Assignee: VOLCANO CORPORATION, San Diego, CA (US)

(21) Appl. No.: 14/135,856

(22) Filed: Dec. 20, 2013

Related U.S. Application Data
(60) Provisional application No. 61/745,449, filed on Dec. 21, 2012.

(51) Int. Cl. A61B 8/12; A61B 8/08

(52) U.S. Cl. CPC A61B 8/12; A61B 8/0841
USPC 600/424; 600/463

(57) ABSTRACT

The invention generally relates to devices and methods that allow an operator to obtain real-time images of a luminal surface prior to, during, and after an intraluminal procedure, including while a tool is engaged with the luminal surface. An imaging system of the invention may include a first elongate member that includes a first imaging element and a catheter that includes a second imaging element. The first imaging element is an optical-to-acoustic transducer, and the second imaging element is an optical-to-acoustic transducer. The catheter also defines a lumen and includes cutting element. The catheter is configured to receive at least a portion of the elongate member within the lumen and is configured to move along a path of the elongate member.

### RELATED APPLICATION

This application claims the benefit of and priority to U.S. Provisional Ser. No. 61/745,449, filed Dec. 21, 2012, which is incorporated by reference in its entirety.

### TECHNICAL FIELD

The present invention generally relates to devices and methods for intraluminal imaging and intraluminal procedures.

### BACKGROUND

Cardiovascular disease frequently arises from the accumulation of atheroma material on inner walls of vascular lumens, particularly arterial lumens of the coronary and other vasculature, resulting in a condition known as atherosclerosis. Atherosclerosis occurs naturally as a result of aging, but it may also be aggravated by factors such as diet, hypertension, heredity, and vascular injury. Atheroma and other vascular deposits restrict blood flow and can cause ischemia that, in acute cases, can result in myocardial infarction. Atheroma deposits can have widely varying properties, with some deposits being relatively soft and others being fibrous and/or calcified. In the latter case, the deposits are frequently referred to as plaque.

Treatment of cardiovascular disease often requires intraluminal imaging and intraluminal interventional therapy. Such intraluminal treatment involves the introduction, movement, and exchange of multiple components, such as guidewires, imaging catheters and interventional catheters, into the delicate vasculature. This risks inadvertent vessel injury and/or further damage to vessels that are often already weakened by the disease.

For example, a guidewire is often advanced through the patient's vasculature along a path suspected of having atheroma within the vessel. Once in place, an imaging catheter is threaded onto the guidewire and urged distally until the imaging catheter reaches the atheroma. If the guidewire is misplaced, the imaging catheter is removed, the guidewire is re-positioned, and the imaging catheter is reintroduced. Once the imaging catheter visually confirms the location of the atheroma, the imaging catheter is exchanged one or more interventional catheters to treat the atheroma. During and/or after the interventional therapy, the imaging catheter may be re-introduced to monitor and evaluate the treatment. Occasionally, the treated vessel may require introduction of a stent to prevent embolization of the treated vessel. If so, another interventional catheter is introduced to place the stent and removed. Thereafter, the imaging catheter is re-introduced to image stent placement.

Locating the region of interest and exchanging the imaging catheter for one or more interventional catheters within a patient's vascular system is time consuming. In addition, the multiple exchanges may be injurious to the patient because the blood vessel interior is delicate, may be weakened by disease, and is therefore susceptible to injury from movement of the catheter body within it. As such, the need to move a catheter, let alone multiple catheters, within the patient should be minimized.

In order to reduce the number of catheter exchanges, some technologies have incorporated an imaging sensor on the interventional catheter. For example, it is known to include an imaging sensor with an atherectomy catheter by locating the imaging sensor proximal or distal to the catheter's removal assembly as described by Radvancy et al. in “Seminars in Interventional Radiology” (25(1), 11-19, 2008), or by integrating the imaging sensor into the removal assembly as indicated in U.S. Pat. No. 7,927,784.

### SUMMARY

The invention recognizes that current intraluminal imaging and interventional techniques do not allow for real-time imaging of the vessel area during the treatment procedure. Rather, current techniques require exchanging multiple catheters or moving a combined imaging/interventional catheter back and forth to alternatively image and treat. In addition, those techniques do not resolve failures to locate a region of interest within a lumen due to a misplaced guidewire. Devices and methods of the invention provide for real-time imaging of a vessel to be treated prior to treatment, during treatment, and after treatment while minimizing the number of catheters that are introduced into the vessel. This reduces risk associated with exchanging and moving multiple catheters within the delicate vasculature that may be weakened by disease. Aspects of the invention are accomplished by providing an imaging system that includes an imaging guidewire and an imaging catheter, which may include one or more intraluminal tools for performing an intraluminal procedure. In one embodiment, the intraluminal tool is an extraction tool that includes one or more cutting elements for cutting and removing atheroma from a luminal surface.

A particular benefit of certain aspects of the invention is that the placement of imaging elements on the guidewire and the catheter allow an operator to obtain real-time images of the vessel wall while the intraluminal tool is engaged with the vessel surface. This increases safety and allows an operator to better direct the intraluminal procedure. In certain embodiments, the imaging system simultaneously provides both distal and side views of the treatment area. For example, imaging elements along the length of the guidewire provide a side view of the intraluminal procedure and imaging elements on a distal end face of the imaging catheter provide a distal view of the intraluminal procedure. Additionally, such placement of the imaging elements eliminates the need to move the catheter back and forth in order to alternate between imaging and treatment, thereby improving overall efficiency of the procedure.

In addition, the imaging guidewire of the invention may obtain real-time images of the luminal surface that allow an operator to initially direct the guidewire into the proper position. Beyond reducing guidewire misplacement, the imaging guidewire can be used to locate and survey the diseased tissue itself. Locating the region of interest with the guidewire reduces risk of trauma to the vessel because the imaging guidewire is significantly smaller than a typical imaging catheter. Furthermore, as the imaging catheter of the invention is driven over the imaging guidewire to the region of interest, the operator can receive real-time images of the vessel from both the guidewire and the catheter to maximize vessel visualization and provide confirmation of the imaging catheter's location with respect to the region of interest. Upon appropriate placement of the imaging catheter, an intraluminal tool can be deployed to perform an intraluminal procedure at the region of interest, while the imaging catheter and imaging catheter provide real-time imaging of the procedure.

In certain aspects, the imaging system includes an elongate member and a catheter. The elongate member includes at least one imaging element, which may be an acoustic-to-optical transducer. In certain embodiments, the first elongate member is a guidewire. The catheter defines a lumen and also includes at least one imaging element. The catheter is configured to receive at least a portion of the elongate member. The second imaging element includes a second acoustic-to-optical transducer.

In order to facilitate intraluminal procedures, the catheter may also be configured to introduce an intraluminal tool and/or a therapeutic device into the lumen. In one embodiment, the intraluminal tool is an extraction tool. The extraction tool includes at least one cutting element. The cutting element is configured to remove an atheroma from a luminal surface for treatment. In order to facilitate removal, the cutting element may be operably associated with a drive shaft to rotate the cutting element. Rotation of drive shaft translates into rotation of the cutting element, which causes the cutting element to morcellate or shave off atheroma material occluding the vessel. However, any non-rotational movement of the cutting element may also be applied to remove atheroma material. In another embodiment, the cutting element is operable associated with an inflatable balloon. In this embodiment, inflation of the balloon causes the cutting element to engage against the luminal surface and movement (longitudinal, rotational, etc.), of the cutting element cuts the atheroma material. The catheter may further include a disposal member that is configured to dispose of the cut or morcellated atheroma material from the lumen. For example, the catheter may include a disposal lumen that is coupled to a vacuum source, which can be used to suction the morcellated atheroma material from the lumen.

In certain embodiments, the imaging elements of elongate member and the catheter are acoustic-to-optical transducers. The acoustic-to-optical transducers are configured to receive acoustic signals reflected from the luminal surface. The received signals can be used to generate an image of the luminal surface. In a further embodiment, the first and second acoustic-to-optical transducers are configured to generate an acoustic signal. The acoustic-to-optical transducers of the elongate member and catheter may be the same or different. In certain embodiments, the acoustic-to-optical transducers include a Fiber Bragg Grating element in an optical fiber. In addition to the acoustic-to-optical transducers, the imaging elements of the elongate member, catheter, or both may include at least one other transducer. The at least one other transducer can be used to generate an acoustic signal to reflect off the luminal surface. The at least one other transducer can be an electrical-to-acoustic transducer or an optical-to-acoustic transducer. In one embodiment, the at least one other transducer is a piezoelectric transducer or a photoacoustic transducer.

Aspects of the invention further include methods for intraluminal imaging and performing an intraluminal procedure. According to one embodiment, the method includes the steps of delivering an elongate member into a lumen, imaging a surface of the lumen with the elongate member to locate atheroma material, guiding the catheter over the elongate member towards the atheroma material, imaging the surface of the lumen, as the catheter is guided towards the atheroma material, to position the catheter for atheroma material removal, and removing the atheroma material from the surface with the cutting element. In certain embodiments, the method further includes simultaneously imaging the cutting element as it removes the atheroma material from luminal surface with the elongate member, catheter, or both. In addition, the method may further include removing the cut or morcellated atheroma material from the lumen.

Other and further aspects and features of the invention will be evident from the following detailed description and accompanying drawings, which are intended to illustrate, not limit, the invention.

### BRIEF DESCRIPTION OF DRAWINGS

FIG. 1 shows an exemplary embodiment of the imaging system.

FIG. 2 depicts an optical fiber suitable for use with the imaging system according to certain embodiments.

FIG. 3 depicts an embodiment of an imaging element that includes a piezoelectric element.

FIGS. 4 and 5 depict an imaging element according to this embodiment that uses Fiber Bragg Gratings to generate acoustic energy.

FIG. 6 is a block diagram generally illustrating an imaging assembly of the invention and several associated interface components.

FIG. 7 is a block diagram illustrating another example of an imaging assembly of the invention and associated interface components.

FIG. 8 shows a cross-section of the imaging guidewire including a plurality of imaging elements according to one embodiment.

FIG. 9 depicts a distal portion of an imaging guidewire according to one embodiment.

FIG. 10 illustrates a cross-sectional view of an imaging catheter according to one embodiment.

FIG. 11 depicts another embodiment of the imaging catheter.

FIG. 12 depicts a cross-sectional view of a lumen of the imaging catheter having a pusher element disposed therein according to one embodiment.

FIGS. 13A- 13C depict an implant delivery mechanism that includes an expansion balloon according to one embodiment.

FIG. 14 shows an angioplasty tool according to one embodiment.

FIG. 15 depicts an ablation tool suitable for use with the imaging catheter of the invention.

FIGS. 16A-16C depict an ablation tool suitable for use with the imaging catheter of the invention.

FIG. 17 depicts an ablation tool suitable for use with the imaging catheter of the invention.

FIG. 18 depicts an ablation tool suitable for use with the imaging catheter of the invention.

FIG. 19 depicts an embodiment of a distal end of an extraction tool.

FIG. 20 depicts an embodiment of a distal end of an extraction tool.

FIG. 21 depicts an embodiment of a distal end of an extraction tool.

FIG. 22 depicts an embodiment of a distal end of an extraction tool.

FIGS. 23A through 23C show some exemplary embodiments of a distal end of an extraction tool 28.

### DETAILED DESCRIPTION

The present invention generally relates to imaging systems and methods for intraluminal imaging that include an imaging guidewire, an imaging catheter, or both. The imaging systems of the invention provide for 1) real-time imaging of intraluminal surfaces to detect a location of interest prior to introduction of a catheter, 2) performing intraluminal procedures at the location of interest, and 3) real-time imaging of the location of interest before, during, and after the intraluminal procedure. Both the imaging guidewire and the imaging catheter of an imaging system may utilize acoustic-to-optical transducers to image the intraluminal surface and lumen. The imaging systems can be used for vascular or nonvascular imaging. The imaging catheter may include a tool element, such an ablation, implant delivery, or extraction device, to perform the intraluminal procedure.

The imaging guidewire of the invention can be introduced into a lumen of the body to obtain real-time images of the vessel prior to introduction of a catheter. The body lumens generally are diseased body lumens and, in particular, lumens of the vasculature. The real-time images obtained may be used to locate a region or location of interest within a body lumen. Regions of interest are typical regions that include a defect. The defect in the body lumen can be a de novo lesion or an in-stent restenosis lesion for example. The devices and methods, however, are also suitable for treating stenosis of body lumens and other hyperplastic and neoplastic conditions in other body lumens, such as the ureter, the biliary duct, respiratory passages, the pancreatic duct, the lymphatic duct, and the like. In addition, the region of interest can include, for example, a location for stent placement or a location including plaque or diseased tissue that needs to be removed.

Once the imaging guidewire is in place, the imaging catheter can be introduced over the guide wire to the location of interest. The imaging catheter can obtain images of the intraluminal surface as the imaging catheter moves towards the region of interest, which allows the imaging catheter to be precisely placed into the region of interest and provides for tracking of the imaging catheter along the path of the guidewire. In addition, the imaging catheter can be used to obtain different imaging views of the region of interest.

In certain aspects, the imaging catheter may also serve as a delivery catheter, ablation catheter, or extraction catheter to perform an intraluminal procedure. The imaging catheter may include a tool element to perform an intraluminal procedure. During the procedure, both the imaging guidewire and the imaging catheter may be used to image cross-sections of the luminal surface. In addition, the imaging catheter may also include forward or distal facing imaging elements to image the luminal space and/or any procedure in front of or distal to the imaging catheter. For example, the imaging guidewire can axially image a stent and luminal surface as it is being deployed distally from the imaging catheter and the imaging catheter can image the lumen proximal to the region of interest to ensure proper catheter placement This greatly improves visualization during the procedure by allowing an operator to have real-time images of the vessel wall while the device or procedure tool is engaged with that portion of the vessel wall. After the procedure, the imaging catheter can be removed from the vessel. The imaging guidewire can be used to perform a final visualization of the luminal surface.

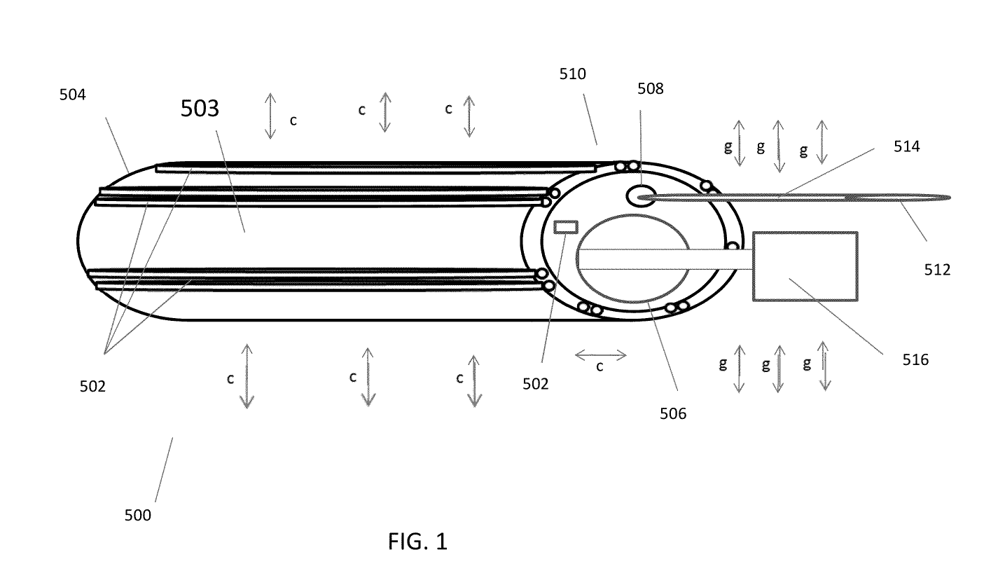

FIG. 1 shows an exemplary embodiment of the imaging system 500. As shown, the imaging system 500 includes an imaging catheter 504 and an imaging guidewire 512. The imaging catheter 504 includes a catheter body 503 and an imaging assembly formed by one or more of imaging elements 502 located on the catheter body 503. The imaging elements 502 located on the length of the catheter body 503 to send and receive imaging signals to image a portion of the luminal surface along the side of the catheter body 503. Imaging elements 502 located on the distal end face 510 of the catheter body 503 are able to image the luminal surface in distal to or in front of the catheter body 503. The c-arrows show the imaging signals of the imaging catheter 504. The imaging catheter 504 defines a guidewire lumen 508 and is configured to receive a guidewire. As shown in FIG. 1, the imaging guidewire 512 runs through and extends distally from the guidewire lumen 508 of the catheter 504.

The imaging guidewire 512 includes at least one imaging element 514. The imaging element 514 of the guidewire 512 can be the same as or different from the imaging elements of the catheter 504. The one or more imaging elements 514 of the guidewire 512 are able to send and receive imaging signals a portion of the luminal surface distal to the catheter body 503. The g-arrows show the imaging signals of the imaging guidewire 512.

Also shown in FIG. 1, the imaging catheter 504 includes a tool lumen 506 and is configured to receive a tool catheter or tool element 516. Through the tool lumen 506, a tool catheter or tool element 516 (e.g. delivery catheter, atherectomy device, ablation device) can be introduced into a vessel to perform an intraluminal procedure. The tool element 516, as shown, is distally deployed from the catheter body 503 and is extended within the imaging signals (g-arrows) of the imaging guidewire 512.

The configuration of the imaging system 500, as shown in FIG. 1, allows an operator to real-time image an intraluminal procedure performed by the tool element 516 with the imaging catheter 504, imaging guidewire 514, or both. Thus, it can be appreciated that the imaging system 500 of the invention greatly increases an operator's ability to view the lumen and luminal surface during a procedure. This enhanced visualization significantly reduces operation time and increases the efficiency of the intraluminal procedure itself. In addition, the imaging system prevents the need to alternate between performing the procedure and obtaining images because both steps can be performed simultaneously.

The imaging guidewire and imaging catheter are configured for intraluminal introduction into a target body lumen. The dimensions and other physical characteristics of the guidewire and catheter will vary significantly depending on the body lumen that is to be accessed. In addition, the dimensions can depend on the placement and amount of imaging elements included in the imaging guidewire or imaging catheter.

For the imaging guidewire, the imaging element can be formed as or be integrated into the body of the imaging guidewire, circumscribe the guidewire, and/or run along the body of the guidewire. The imaging guidewire may also include an outer support structure or coating surrounding the imaging elements. The imaging guidewire including the imaging element (that is, the optical fiber and transducer material) and, in certain embodiments, the surrounding support structure can have a total outside diameter of less than 1 mm, preferably less than 300 micron (less than about 1 French).

Imaging guidewire bodies may include a solid metal or polymer core. Suitable polymers include polyvinylchloride, polyurethanes, polyesters, polytetrafluoroethylenes (PTFE), silicone rubbers, natural rubbers, and the like. Preferably, at least a portion of the metal or polymer core and other elements that form the imaging guidewire body are flexible.

For the imaging catheter, the imaging element can form or be integrated within the body of the catheter, circumscribe the catheter, placed on a distal end face of the catheter, and/or run along the body of the catheter. The imaging catheter may also include an outer support structure or coating surrounding the imaging elements. Imaging catheter bodies intended for intravascular introduction will typically have a length in the range from 50 cm to 200 cm and an outer diameter in the range from 1 French to 12 French (0.33 mm: 1 French), usually from 3 French to 9 French. In the case of coronary catheters, the length is typically in the range from 125 cm to 200 cm, the diameter is preferably below 8 French, more preferably below 7 French, and most preferably in the range from 2 French to 7 French.

Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques. Preferably, at least a portion of the catheter body is flexible.

The imaging catheter and the imaging guidewire of the invention include an imaging assembly. Any imaging assembly may be used with devices and methods of the invention, such as optical-acoustic imaging apparatus, intravascular ultrasound (IVUS) or optical coherence tomography (OCT). The imaging assembly is used to send and receive signals to and from the imaging surface that form the imaging data.

In some embodiments, the imaging assembly is an IVUS imaging assembly. The imaging assembly can be a phased-array IVUS imaging assembly, a pull-back type IVUS imaging assembly, including rotational IVUS imaging assemblies, or an IVUS imaging assembly that uses photoacoustic materials to produce diagnostic ultrasound and/or receive reflected ultrasound for diagnostics. IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. All of these references are incorporated by reference herein in their entirety.

IVUS imaging is widely used in interventional cardiology as a diagnostic tool for assessing a diseased vessel, such as an artery, within the human body to determine the need for treatment, to guide an intervention, and/or to assess its effectiveness. An IVUS device including one or more ultrasound transducers is introduced into the vessel and guided to the area to be imaged. The transducers emit and then receive backscattered ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a 360 degree cross-sectional image of the vessel where the device is placed.

There are two general types of IVUS devices in use today: rotational and solid-state (also known as synthetic aperture phased array). For a typical rotational IVUS device, a single ultrasound transducer element is located at the tip of a flexible driveshaft that spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer. Suitable rotational IVUS catheters include, for example the REVOLUTION 45 MHz catheter (offered by the Volcano Corporation).

In contrast, solid-state IVUS devices carry a transducer complex that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receive sets, the solid-state IVUS system can synthesize the effect of a mechanically scanned transducer element but without moving parts. The same transducer elements can be used to acquire different types of intravascular data. The different types of intravascular data are acquired based on different manners of operation of the transducer elements. The solid-state scanner can be wired directly to the imaging system with a simple electrical cable and a standard detachable electrical connector.

The transducer subassembly can include either a single transducer or an array. The transducer elements can be used to acquire different types of intravascular data, such as flow data, motion data and structural image data. For example, the different types of intravascular data are acquired based on different manners of operation of the transducer elements. For example, in a gray-scale imaging mode, the transducer elements transmit in a certain sequence one gray-scale IVUS image. Methods for constructing IVUS images are well-known in the art, and are described, for example in Hancock et al. (U.S. Pat. No. 8,187,191), Nair et al. (U.S. Pat. No. 7,074,188), and Vince et al. (U.S. U.S. Pat. No. 6,200,268), the content of each of which is incorporated by reference herein in its entirety. In flow imaging mode, the transducer elements are operated in a different way to collect the information on the motion or flow. This process enables one image (or frame) of flow data to be acquired. The particular methods and processes for acquiring different types of intravascular data, including operation of the transducer elements in the different modes (e.g., gray-scale imaging mode, flow imaging mode, etc.) consistent with the present invention are further described in U.S. patent application Ser. No. 14/037,683, the content of which is incorporated by reference herein in its entirety.

The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety. Commercially available fluid flow display software for operating an IVUS catheter in flow mode and displaying flow data is CHROMAFLOW (IVUS fluid flow display software offered by the Volcano Corporation).

Suitable phased array imaging catheters include Volcano Corporation's EAGLE EYE Platinum Catheter, EAGLE EYE Platinum Short-Tip Catheter, and EAGLEEYE Gold Catheter.

The imaging guidewire of the present invention may also include advanced guidewire designs to include sensors that measure flow and pressure, among other things. For example, the FLOWIRE Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. Additionally, the PrimeWire PRESTIGE pressure guidewire, available from Volcano Corp. (San Diego, Calif.), provides a microfabricated microelectromechanical (MEMS) pressure sensor for measuring pressure environments near the distal tip of the guidewire. Additional details of guidewires having MEMS sensors can be found in U.S. Patent Publication No. 2009/0088650, incorporated herein by reference in its entirety. In addition to IVUS, other intraluminal imaging technologies may be suitable for use in methods of the invention for assessing and characterizing vascular access sites in order to diagnose a condition and determine appropriate treatment. For example, an Optical Coherence Tomography catheter may be used to obtain intraluminal images in accordance with the invention.

OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In OCT, a light source delivers a beam of light to an imaging device to image target tissue. Light sources can include pulsating light sources or lasers, continuous wave light sources or lasers, tunable lasers, broadband light source, or multiple tunable laser. Within the light source is an optical amplifier and a tunable filter that allows a user to select a wavelength of light to be amplified. Wavelengths commonly used in medical applications include near-infrared light, for example between about 800 nm and about 1700 nm.

Aspects of the invention may obtain imaging data from an OCT system, including OCT systems that operate in either the time domain or frequency (high definition) domain. Basic differences between time-domain OCT and frequency-domain OCT is that in time-domain OCT, the scanning mechanism is a movable minor, which is scanned as a function of time during the image acquisition. However, in the frequency-domain OCT, there are no moving parts and the image is scanned as a function of frequency or wavelength.

In time-domain OCT systems an interference spectrum is obtained by moving the scanning mechanism, such as a reference minor, longitudinally to change the reference path and match multiple optical paths due to reflections within the sample. The signal giving the reflectivity is sampled over time, and light traveling at a specific distance creates interference in the detector. Moving the scanning mechanism laterally (or rotationally) across the sample produces two-dimensional and three-dimensional images.

In frequency domain OCT, a light source capable of emitting a range of optical frequencies excites an interferometer, the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics letters, Vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics 28: 3339-3342), wherein the distance to a scatterer is determined by the wavelength dependent fringe spacing within the power spectrum. SD-OCT has enabled the determination of distance and scattering intensity of multiple scatters lying along the illumination axis by analyzing a single the exposure of an array of optical detectors so that no scanning in depth is necessary. Typically the light source emits a broad range of optical frequencies simultaneously.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Generally, time domain systems and frequency domain systems can further vary in type based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in its entirety.

In certain embodiments, angiogram image data is obtained simultaneously with the imaging data obtained from the imaging catheter and/or imaging guidewire of the present invention. In such embodiments, the imaging catheter and/or guidewire may include one or more radiopaque labels that allow for co-locating image data with certain positions on a vasculature map generated by an angiogram. Co-locating intraluminal image data and angiogram image data is known in the art, and described in U.S. Publication Nos. 2012/0230565, 2011/0319752, and 2013/0030295.

In preferred embodiments, the imaging assembly is an optical-acoustic imaging apparatus. Optical-acoustic imaging apparatus include at least one imaging element to send and receive imaging signals. In one embodiment, the imaging element includes at least one acoustic-to-optical transducer. In certain embodiments, the acoustic-to-optical transducer is an Fiber Bragg Grating within an optical fiber. In addition, the imaging elements may include the optical fiber with one or more Fiber Bragg Gratings (acoustic-to-optical transducer) and one or more other transducers. The at least one other transducer may be used to generate the acoustic energy for imaging. Acoustic generating transducers can be electric-to-acoustic transducers or optical-to-acoustic transducers. The imaging elements suitable for use in devices of the invention are described in more detail below.

Fiber Bragg Gratings for imaging provides a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interferences in the beam. If the paths are identical, then the two paths combine to form the original beam. If the paths are different, then the two parts will add or subtract from each other and form an interference. The Fiber Bragg Grating elements are thus able to sense a change wavelength between the constant path and the change path based on received ultrasound or acoustic energy. The detected optical signal interferences can be used to generate an image using any conventional means.

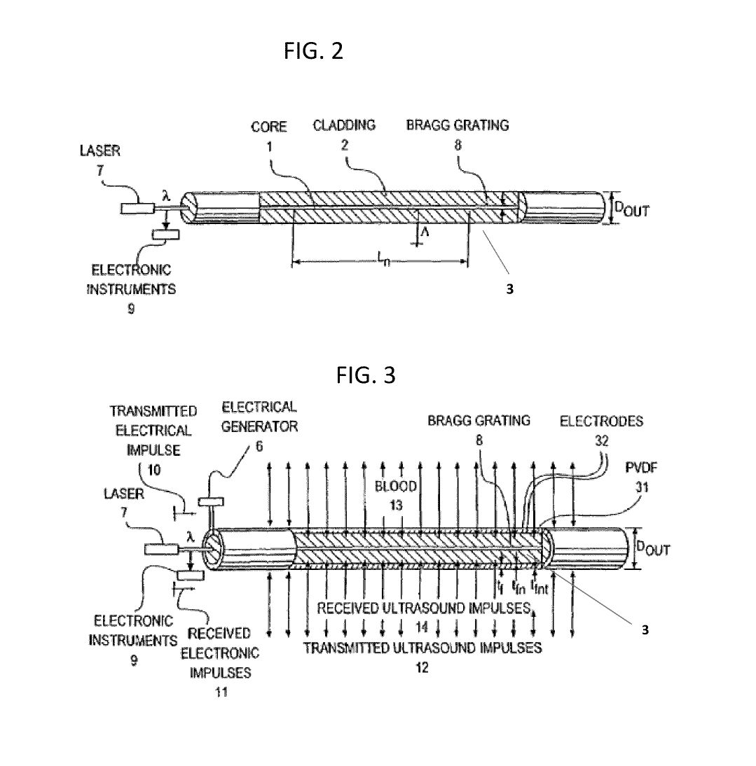

FIG. 2 depicts an optical fiber 3 for use with an imaging element according to certain embodiments. The optical fiber 3 may be a single mode optical fiber. The optical fiber 3 includes a core 1, a cladding 2, and a Fiber Bragg Grating 8. The optical fiber 3 is coupled includes a laser 7. The Bragg Grating 8 will reflect back a narrowband component centered about the Bragg wavelength λ given by λ=2Λ, where n is the index of the core of the fiber and A represents the grating period. With a tunable laser 7 and different grating periods (each period at approximately 0.5μ) at different positions on the fiber, it is possible to make independent measurements in each of the grating positions. As used in the imaging guidewire and imaging catheter of the invention, the optical fiber 3 with Fiber Bragg Grating 8 acts as an acoustic-to-optical transducer.

In certain embodiments, the imaging element includes a piezoelectric element to generate the acoustic or ultrasound energy. In such aspect, the optical fiber of the imaging element may by coated by the piezoelectric element. The piezoelectric element may include any suitable piezoelectric or piezoceramic material. In one embodiment, the piezoelectric element is a poled polyvinylidene fluoride or polyvinylidene difluoride material. The piezoelectric element can be connected to one or more electrodes that are connected to a generator that transmits pulses of electricity to the electrodes. The electric pulses cause mechanical oscillations in the piezoelectric element, which generates an acoustic signal. Thus, the piezoelectric element is an electric-to-acoustic transducer. Primary and reflected pulses (i.e. reflected from the imaging medium) are received by the Bragg Grating element and transmitted to an electronic instrument to generate an imaging.

FIG. 3 depicts an embodiment of an imaging element that includes a piezoelectric element. The imaging element includes an optical fiber 3 (such as the optical fiber in FIG. 2) with Fiber Bragg Grating 8 and a piezoelectric element 31. As shown in FIG. 3, an electrical generator 6 stimulates the piezoelectric element 31 (electrical-to-acoustic transducer) to transmit ultrasound impulses 10 to both the Fiber Bragg Grating 8 and the outer medium 13 in which the device is located. For example, the outer medium may include blood when imaging a vessel. Primary and reflected impulses 11 are received by the Fiber Bragg Grating 8 (acting as an acoustic-to-optical transducer). The mechanical impulses deform the Bragg Grating and cause the Fiber Bragg Grating to modulate the light reflected within the optical fiber, which generates an interference signal. The interference signal is recorded by electronic detection instrument 9, using conventional methods. The electronic instrument may include a photodetector and an oscilloscope. An image can be generated from these recorded signals. The electronic instruments 9 modulation of light reflected backwards from the optical fiber due to mechanical deformations. The optical fiber with a Bragg Grating described herein and shown in FIG. 2, the imaging element described herein and shown in FIG. 3 and other varying embodiments are described in more detail in U.S. Pat. Nos. 6,659,957 and 7,527,594 and in U.S. Patent Publication No. 2008/0119739.

In another aspect, the imaging element does not require an electrical-to-acoustic transducer to generate acoustic/ultrasound signals. Instead, the imaging element utilizes the one or more Fiber Bragg Grating elements of the optical fiber in combination with an optical-to-acoustic transducer material to generate acoustic energy from optical energy. In this aspect, the acoustic-to-optical transducer (signal receiver) also acts as an optical-to-acoustic transducer (signal generator).

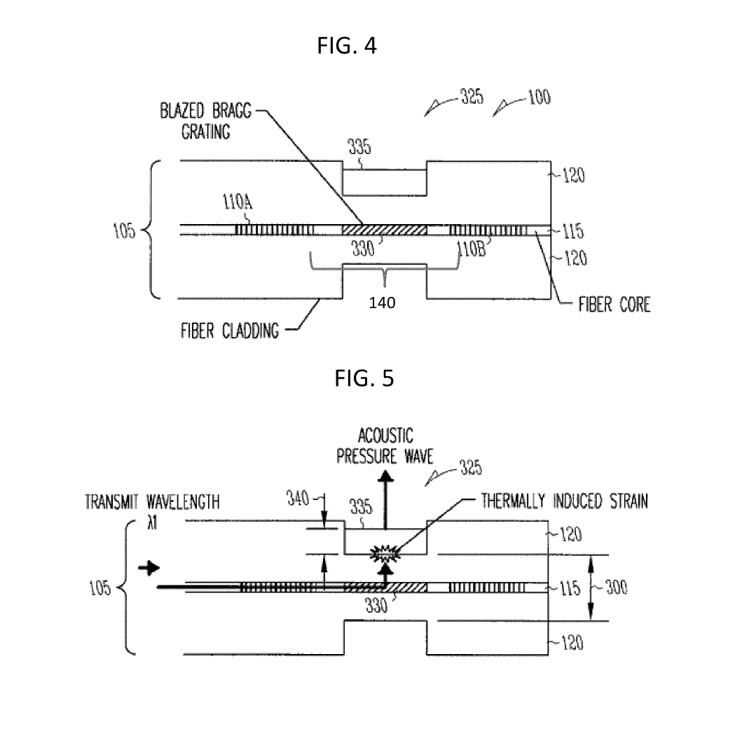

To generate the acoustic energy, imaging element may include a combination of blazed and unblazed Fiber Bragg Gratings. Unblazed Bragg Gratings typically include impressed index changes that are substantially perpendicular to the longitudinal axis of the fiber core of the optical fiber. Unblazed Bragg Gratings reflect optical energy of a specific wavelength along the longitudinal of the optical fiber. Blazed Bragg Gratings typically include obliquely impressed index changes that are at a non-perpendicular angle to the longitudinal axis of the optical fiber. Blazed Bragg Gratings reflect optical energy away from the longitudinal axis of the optical fiber. FIGS. 4 and 5 depict an imaging element according to this embodiment.

FIG. 4 shows an example of imaging element that uses Fiber Bragg Gratings to generate acoustic energy. As depicted in FIG. 4, the imaging element 100 includes an optical fiber 105 with unblazed Fiber Bragg Grating 110A and 110B and blazed Fiber Bragg Grating 330 and a photoacoustic material 335 (optical-to-acoustic transducer). The region between the unblazed Fiber Bragg Grating 110A and 110B is known as the strain sensing region 140. The strain sensing region may be, for example, 1 mm in length. The Blazed Fiber Bragg Grating 330 is implemented in the strain sensing region 140. The photoacoustic material 335 is positioned to receive the reflected optical energy from the blazed Fiber Bragg Grating 330. Although not shown, the proximal end of the optical fiber 105 is operably coupled to a laser and one or more electronic detection elements.

In operation and as depicted in FIG. 5, the blazed Fiber Bragg Grating 330 receives optical energy of a specific wavelength λ1 from a light source, e.g. a laser, and blazed Grating 330 directs that optical energy towards photoacoustic material 335. The received optical energy in the photoacoustic material 335 is converted into heat, which causes the material 335 to expand. Pulses of optical energy sent to the photoacoustic material 335 cause the photoacoustic material 335 to oscillate. The photoacoustic material 335 oscillates, due to the received optical energy, at a pace sufficient to generate an acoustic or ultrasound wave. The acoustic wave is transmitted and reflected from the imaging surface and reflected back to the imaging element. The acoustic wave reflected from the imaging surface impinges on photoacoustic transducer 335, which causes a vibration or deformation of photoacoustic transducer 335. This results in a change in length of light path within the strain sensing region 140. Light received by blazed fiber Bragg grating from photoacoustic transducer 135 and into fiber core 115 combines with light that is reflected by either fiber Bragg grating 110A or 110B (either or both may be including in various embodiments). The light from photoacoustic transducer 135 will interfere with light reflected by either fiber Bragg grating 110A or 110B and the light returning to the control unit will exhibit an interference pattern. This interference pattern encodes the ultrasonic image captured by imaging element 100. The light 137 can be received into photodiodes within a control unit and the interference pattern thus converted into an analog electric signal. This signal can then be digitized using known digital acquisition technologies and processed, stored, or displayed as an image of the target treatment site.

Acoustic energy of a specific frequency may be generated by optically irradiating the photoacoustic material 335 at a pulse rate equal to the desired acoustic frequency. The photoacoustic material 335 can be any suitable material for converting optical energy to acoustic energy and any suitable thickness to achieve a desired frequency. The photoacoustic material 335 may have a coating or be of a material that receives acoustic energy over a band of frequencies to improve the generation of acoustic energy by the photoacoustic material and reception of the acoustic energy by the optical fiber sensing region.

In one example, the photoacoustic material 335 has a thickness 340 (in the direction in which optical energy is received from blazed Bragg grating 330) that is selected to increase the efficiency of emission of acoustic energy. In one example, thickness 340 is selected to be about ¼ the acoustic wavelength of the material at the desired acoustic transmission/reception frequency. This improves the generation of acoustic energy by the photoacoustic material.

In a further example, the photoacoustic material is of a thickness 300 that is about ¼ the acoustic wavelength of the material at the desired acoustic transmission/reception frequency, and the corresponding glass-based optical fiber sensing region resonant thickness 300 is about ½ the acoustic wavelength of that material at the desired acoustic transmission/reception frequency. This further improves the generation of acoustic energy by the photoacoustic material and reception of the acoustic energy by the optical fiber sensing region. A suitable photoacoustic material is pigmented polydimethylsiloxane (PDMS), such as a mixture of PDMS, carbon black, and toluene.

The imaging element described and depicted in FIGS. 4 and 5 and other varying embodiments are described in more detail in U.S. Pat. Nos. 7,245,789, 7447,388, 7,660,492, 8,059,923 and in U.S. Patent Publication Nos. 2010/0087732 and 2012/0108943.

In certain embodiments, an optical fiber of an imaging element (such as one shown in FIGS. 3-5) can include a plurality of Fiber Bragg Gratings, each with its own unique period (e.g. 0.5μ), that interact with at least one other transducer. Because each Fiber Bragg Grating can be directed to transmit and receive signals of specific wavelengths, the plurality of Fiber Bragg Gratings in combination with a tunable filter can be used to generate an array of distributed sonars.

One or more imaging elements may be incorporated into an imaging guidewire or imaging catheter to allow an operator to image a luminal surface. The one or more imaging elements of the imaging guidewire or catheter are referred to generally as an imaging assembly

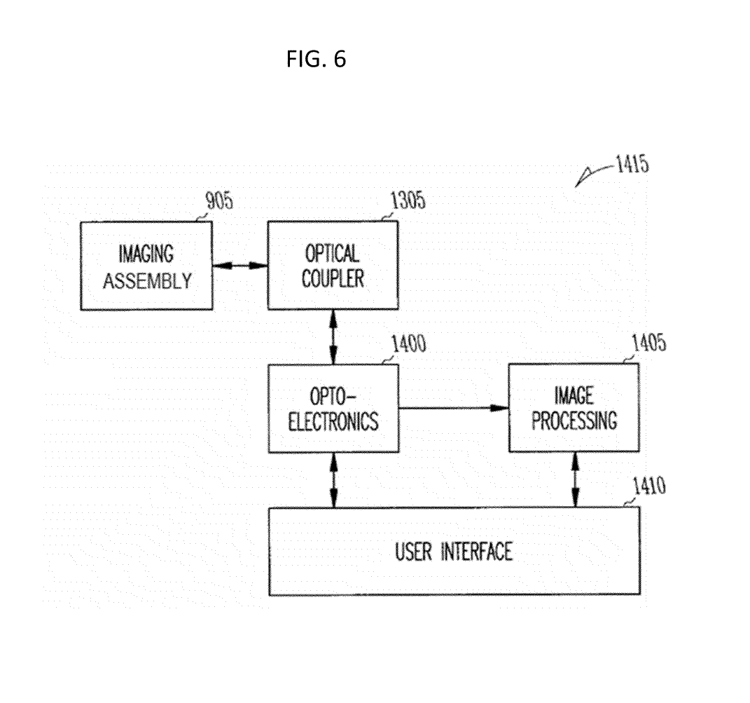

FIG. 6 is a block diagram illustrating generally an imaging assembly 905 and several associated interface components. The block diagram of FIG. 6 includes the imaging assembly 905 that is coupled by optical coupler 1305 to an optoelectronics module 1400. The optoelectronics module 1400 is coupled to an image processing module 1405 and a user interface 1410 that includes a display providing a viewable still and/or video image of the imaging region near one or more acoustic-to-optical transducers using the acoustically-modulated optical signal received therefrom. In one example, the system 1415 illustrated in the block diagram of FIG. 26 uses an image processing module 1405 and a user interface 1410 that are substantially similar to existing acoustic imaging systems.

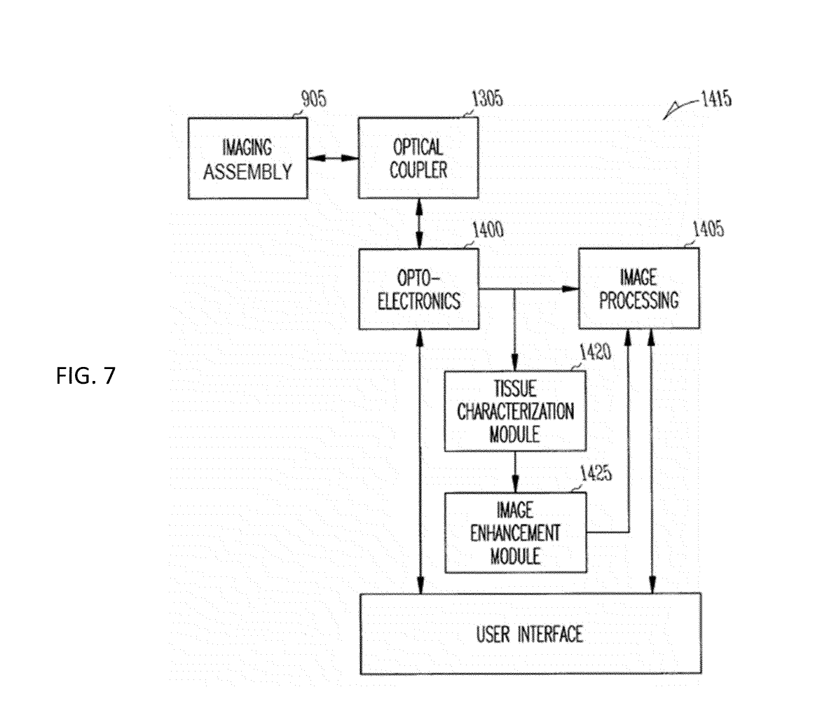

FIG. 7 is a block diagram illustrating generally another example of the imaging assembly 905 and associated interface components. In this example, the associated interface components include a tissue (and plaque) characterization module 1420 and an image enhancement module 1425. In this example, an input of tissue characterization module 1420 is coupled to an output from optoelectronics module 1400. An output of tissue characterization module 1420 is coupled to at least one of user interface 1410 or an input of image enhancement module 1425. An output of image enhancement module 1425 is coupled to user interface 1410, such as through image processing module 1405.

In this example, tissue characterization module 1420 processes a signal output from optoelectronics module 1400. In one example, such signal processing assists in distinguishing plaque from nearby vascular tissue. Such plaque can be conceptualized as including, among other things, cholesterol, thrombus, and loose connective tissue that build up within a blood vessel wall. Calcified plaque typically reflects ultrasound better than the nearby vascular tissue, which results in high amplitude echoes. Soft plaques, on the other hand, produce weaker and more texturally homogeneous echoes. These and other differences distinguishing between plaque deposits and nearby vascular tissue are detected using tissue characterization signal processing techniques.

For example, such tissue characterization signal processing may include performing a spectral analysis that examines the energy of the returned ultrasound signal at various frequencies. A plaque deposit will typically have a different spectral signature than nearby vascular tissue without such plaque, allowing discrimination therebetween. Such signal processing may additionally or alternatively include statistical processing (e.g., averaging, filtering, or the like) of the returned ultrasound signal in the time domain. Other signal processing techniques known in the art of tissue characterization may also be applied. In one example, the spatial distribution of the processed returned ultrasound signal is provided to image enhancement module 1425, which provides resulting image enhancement information to image processing module 1405. In this manner, image enhancement module 1425 provides information to user interface 1410 that results in a displaying plaque deposits in a visually different manner (e.g., by assigning plaque deposits a discernible color on the image) than other portions of the image. Other image enhancement techniques known in the art of imaging may also be applied. In a further example, similar techniques are used for discriminating between vulnerable plaque and other plaque, and enhancing the displayed image provides a visual indicator assisting the user in discriminating between vulnerable and other plaque.

The opto-electronics module 1400 may include one or more lasers and fiber optic elements. In one example, such as where different transmit and receive wavelengths are used, a first laser is used for providing light to the imaging assembly 905 for the transmitted ultrasound, and a separate second laser is used for providing light to the imaging assembly 905 for being modulated by the received ultrasound. In this example, a fiber optic multiplexer couples each channel (associated with a particular one of the optical fibers 925) to the transmit and receive lasers and associated optics. This reduces system complexity and costs.

In one example, the sharing of transmission and reception components by multiple guidewire channels is possible at least in part because the acoustic image is acquired over a relatively short distance (e.g., millimeters). The speed of ultrasound in a human or animal body is slow enough to allow for a large number of transmit/receive cycles to be performed during the time period of one image frame. For example, at an image depth (range) of about 2 cm, it will take ultrasonic energy approximately 26 microseconds to travel from the sensor to the range limit, and back. In one such example, therefore, an about 30 microseconds transmit/receive (T/R) cycle is used. In the approximately 30 milliseconds allotted to a single image frame, up to 1,000 T/R cycles can be carried out. In one example, such a large number of T/R cycles per frame allows the system to operate as a phased array even though each sensor is accessed in sequence. Such sequential access of the photoacoustic sensors in the guidewire permits (but does not require) the use of one set of T/R opto-electronics in conjunction with a sequentially operated optical multiplexer.

In one example, instead of presenting one 2-D slice of the anatomy, the system is operated to provide a 3-D visual image that permits the viewing of a desired volume of the patient's anatomy or other imaging region of interest. This allows the physician to quickly see the detailed spatial arrangement of structures, such as lesions, with respect to other anatomy.

In one example, in which the imaging assembly 905 includes 30 sequentially-accessed optical fibers having up to 10 photoacoustic transducer windows per optical fiber, 30×10=300 T/R cycles are used to collect the image information from all the openings for one image frame. This is well within the allotted 1,000 such cycles for a range of 2 cm, as discussed above. Thus, such an embodiment allows substantially simultaneous images to be obtained from all 10 openings at of each optical fiber at video rates (e.g., at about 30 frames per second for each transducer window). This allows real-time volumetric data acquisition, which offers a distinct advantage over other imaging techniques. Among other things, such real-time volumetric data acquisition allows real-time 3-D vascular imaging, including visualization of the topology of a blood vessel wall, the extent and precise location of plaque deposits, and, therefore, the ability to identify vulnerable plaque.

In certain aspects, one or more imaging elements are incorporated into an imaging guidewire. The imaging guidewire of the invention allows one to image a luminal surface prior to introducing an imaging catheter into the body lumen, such as a blood vessel. Because the imaging guidewire obtains images of the luminal surface, an operator can use the imaging guidewire to find a region of interest within the vasculature prior to introducing a catheter device. The one or more imaging elements can be formed around an inner guidewire body, integrated into an inner guidewire body, or form the guidewire body itself. The imaging guidewire may include a support structure covering at least a portion of the imaging element. The support structure can include one or more imaging windows that allow the imaging element to send and receive signals that form the imaging data.

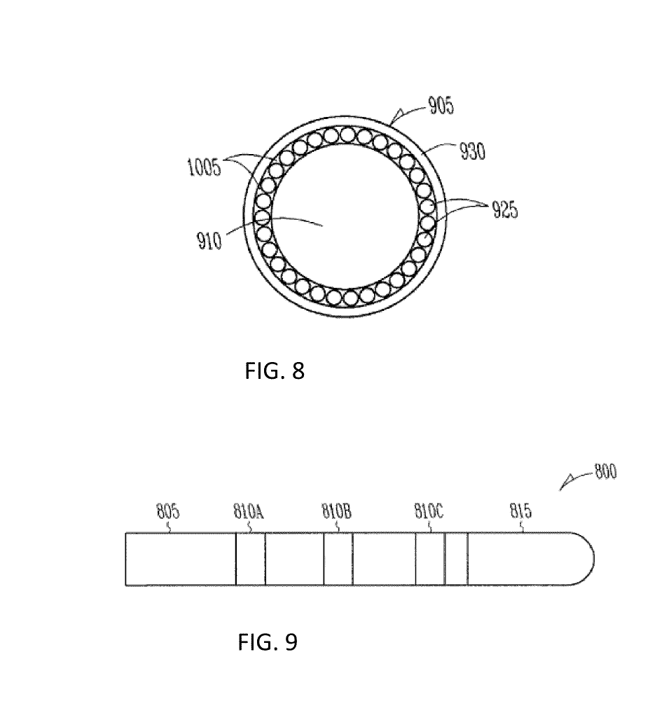

In one example, a plurality of imaging elements surrounds an inner guidewire body. FIG. 8 shows a cross-section of the imaging guidewire 905 showing a plurality of imaging elements surrounding the inner guidewire body 910. The imaging elements 925 are placed next to each other, parallel to, and along the length of the inner guidewire body 910. The guidewire body 910 can be any suitable flexible material. A binder material 1005 can provide structure support to the imaging elements 925. The imaging elements 925 are optionally overlaid with a protective outer coating 930 that provides for transmission of imaging signals.

Typically, the imaging elements are placed parallel to and along the length of the guidewire. In such aspect, the imaging elements image surfaces substantially perpendicular to the longitudinal axis of the imaging guidewire. However, other configurations may be used. For example, one or more imaging elements may be wrapped around the inner guidewire body. In addition, it is also contemplated at least a portion of the imaging elements are positioned substantially across the longitudinal axis of the guidewire. For example, the imaging elements can be positioned across a distal tip of the imaging guidewire such that the imaging elements image objects or surfaces in front of the imaging guidewire. This position of the imaging elements is described in more detail in co-owned and co-pending application entitled “Chronic Total Occlusion Catheter.”

In certain embodiments, the imaging guidewire further includes a support structure surrounding the one or more imaging elements. The support structure may include a plurality of imaging windows to allow transmission and reception of imaging signals (e.g. acoustic signals). FIG. 9 depicts a distal portion 800 of an imaging guidewire 805 according to one embodiment. The imaging guidewire 805 includes one or more imaging windows 810A, 810B, . . . , 810N. Each imaging window 810 may expose at least a portion of one or more imaging elements. The exposed portion of each imaging element may include one or more acoustic-to-optical transducers (e.g. Fiber Bragg Grating in an optical fiber) that correspond to one or more optical-to-acoustic transducers (i.e. photoacoustic material) or one or more electrical-to-acoustic transducers (i.e. piezoelectric material).

The imaging guidewire of the invention may be used in conjunction with an imaging catheter of the invention or any other catheter available. Furthermore, the imaging catheter of the current invention is suitable for use with any other guidewire available. The various embodiments of the imaging guidewire can be used in combination with any one of the embodiments of the imaging catheter without limitation. Various embodiments of the imaging catheter are described hereinafter. In addition, it is also contemplated that the various features of the imaging catheter can be combined without limitation.

The imaging catheter allows an operator to image the luminal surface as the catheter is slideably moved along the imaging guidewire to the location of interest. In certain embodiments, the imaging catheter is a combination catheter that can perform intraluminal procedures such as delivering implants, ablation, and extraction.

Like the imaging guidewire, the imaging catheter includes one or more imaging elements. As discussed previously, each imaging element includes one or more acoustic-to-optical transducers (e.g. Fiber Bragg Grating in an optical fiber) that corresponds to one or more optical-to-acoustic transducers (photoacoustic material) or one or more acoustic-to-optical transducers (piezoelectric material). Like the imaging guidewire, the imaging elements can be positioned anywhere along and on the inner body of the imaging catheter.

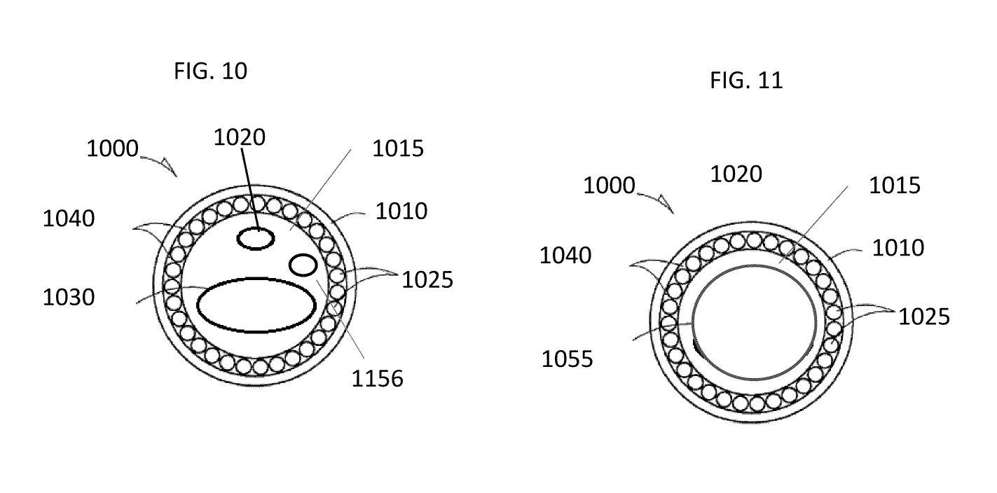

For example, FIG. 10 illustrates a cross-sectional view of an imaging catheter 1000 according to one embodiment. The imaging catheter 1000 includes imaging elements 1025 that surround an inner body member 1015 of the imaging catheter 1000. The imaging elements 1025 are positioned next to each other, parallel to, and along the length of the inner body member 1015. As shown in the cross-sectional view, the imaging elements 1025 are arranged around the circumference of the inner body member 1015 of the imaging catheter 1000. The imaging elements 1025 are disposed in binding material 1040. The imaging catheter 1000 may be surrounded by an outer catheter sheath or protective coating 1010. The outer catheter sheath or protective coating 1010 can be made from any acoustically transparent resiliently flexible material such as polyethylene or the like, which will permit such transparency while maintaining a sterile barrier around the imaging elements.

Further shown in FIG. 10, the imaging catheter 1000 includes a guidewire lumen 1020. The guidewire lumen 1020 receives at least a portion of a guidewire, such as the imaging guidewire. The imaging catheter 1000 can be designed as an over-the-wire catheter or a rapid exchange catheter. Over-the-wire catheters include a guidewire lumen that runs the full length of the catheter. Rapid exchange catheters include a guidewire lumen extending only through a distal portion of the catheter. With respect to the remaining proximal portion of the catheter, the guidewire exits the internal catheter lumen through a guidewire exit port, and the guidewire extends in parallel along the proximal catheter portion.

The imaging catheter 1000 may optionally, and as shown in FIG. 10, include one or more tool lumens 1030. The tool lumen 1030 is formed from an inner catheter sheath or member that is disposed within the inner body 1015 of the imaging catheter 1000. Through the tool lumen 1030, a catheter tool or device can be introduced into a body lumen, such as blood vessel, for treatment. In addition, the imaging catheter may optionally include a removal lumen 1056 that extends from the distal end of the imaging catheter to an opening operably associated with a vacuum source. During intraluminal procedures, a tool element may shave off plaque or other substances from the vessel wall that needs to be removed from the lumen. The shaved-off plaque can be removed from the removal lumen.

FIG. 11 depicts another embodiment of the imaging catheter 1000. In this embodiment, the imaging catheter includes a combined lumen 1055 for receiving the catheter tool or device and the imaging guidewire. The combined lumen 1055 is helpful when the catheter tool or device must also circumscribe the guidewire. For example, implants placed within a body vessel and implant delivery mechanisms are often driven over the guidewire so that the implant may be placed flush against the vessel without the guidewire obstructing implant placement.

Various catheter tools and devices of the imaging catheter are described hereinafter.

In certain aspects, the imaging catheter includes an implant delivery mechanism. The implant delivery mechanism is configured to deploy an implant into the lumen of a body vessel, such as a blood vessel. Often treatment of the vasculature requires placement of an implant or another device into a blood vessel. The implant or device may be placed in the vessel permanently/long term or temporarily/short term purposes. Implants can be placed at the treatment site (such as stents) or implants can be placed near the treatment site to occlude or filter the vessel (such as plugs or filters). For either case, it is desirable to image the implantation site both prior to, during, and after implantation. For example, using the imaging system of the invention, the imaging guidewire can locate the implant placement site and images from both the imaging guidewire and imaging catheter can be used to position the catheter for implant delivery. During implant delivery, the imaging guidewire can image the stent as being deployed distally from the guidewire. For example, imaging during implantation allows an operator to precisely place the implant into position and allows an operator to survey the apposition of the implant after placement. In addition, a combined imaging and delivery catheter prevents the need to exchange a delivery catheter for an imaging catheter, thus decreasing operation time.

The implant delivery mechanism may include a pusher element or inner catheter member with a balloon element configured to deploy an implant out of the imaging catheter. Various embodiments of the implant delivery mechanism are described hereinafter. Each of the described embodiments of the implant delivery mechanism may include a guidewire lumen configured to receive at least a portion of the imaging guidewire. Likewise, implants suitable for use with the imaging catheter may also be configured to receive at least a portion of the imaging guidewire. This allows the implant to be placed within a vessel without the guidewire obstructing the implant and allows the imaging guidewire to image the implant placement from inside the implant. If a balloon element is required for implant placement, the balloon element can be made of an ultrasound-compatible material that allows the imaging guidewire to image stent placement through the balloon.

In one embodiment, the implant delivery mechanism of the imaging catheter includes a pusher element for deploying the implant into the vessel. Any pusher element capable of slidably moving the implant within and out of the tool lumen or combined lumen of the imaging catheter is suitable for use. Typically, the pusher element may be used to deploy self-expanding implants (i.e. implants that do not require balloon expansion). The pusher element is at least partially disposed within the tool lumen of the imaging catheter. The pusher element can be made from a flexible hypotube or wire. Preferably, the pusher element defines a lumen for receiving at least a portion of the guidewire there through to prevent the guidewire from interfering with implant deployment. The distal end of the pusher element may be configured to releasably engage with an implant. For example, the distal end of the pusher element may include flat surface for pushing the implant or the end may include grasping elements that grip the implant as the pusher element drives the implant out of the tool lumen and release the implant into the vessel. Ideally, the distal end of the pusher element provides enough structure and support to deploy the implant through the imaging catheter. In one embodiment, the end of the pusher wire forms a cup that releasably engages with an end portion of the implant.

For implant deployment, the pusher element is moved distally within the tool lumen, thereby driving the implant forward within a stationary imaging catheter. The pusher element continues to move within the lumen until the implant is pushed out of an opening of the tool lumen and into the vessel. In certain embodiments, an actuator associated with the pusher element. The actuator is configured to apply force to the pusher element in order to distally move the pusher element. Once the implant is deployed, the pusher element can be retracted back into the imaging catheter. In preferred embodiments, an imaging guidewire extends distally from a lumen of the pusher element and is able to image the implant as it is deployed out of the pusher element and placed into the lumen.

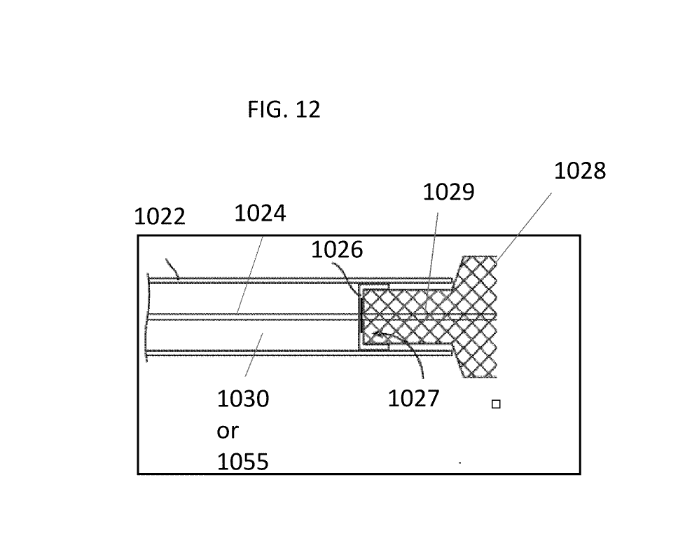

FIG. 12 depicts a side view of a lumen (tool lumen 1030 or combined lumen 1055) of the imaging catheter having a pusher element disposed therein according to one embodiment. As shown in FIG. 12, a pusher element 1024 includes cup 1026. The cup of the pusher element is sized to slideably fit against the surface or sheath 1022 of the tool lumen 1030. The cup 1026 contains an end portion 1027 of the implant 1028. An imaging guidewire 1029 extends distally out of the lumen of the pusher element and extends through a lumen of the implant 1028. This makes sure the guidewire 1029 does not interfering with implant 1028 deployment/expansion and allows the guidewire 1029 to image implant deployment. As shown, the implant 1028 is expandable and the partially deployed out of an opening 1025 of the tool lumen 1030. As the implant 1028 deploys from the tool lumen 1030 of the imaging catheter, the deployed portion of the implant 1028 expands against the vessel walls as it is deployed.

Implants may require expansion for placement into the vessel, and such implants may be self-expandable or require balloon expansion. In some cases, implants may require balloon expansion. As such, certain embodiments of the implant delivery mechanism includes an inflatable delivery balloon. For example, the implant delivery mechanism may include an inner catheter element or pusher element operably associated with an inflatable balloon. The inner catheter element defines an inflatable balloon lumen in which fluid or air can be introduced to inflate the balloon. An implant, such as stent, can be placed over the balloon. The inner catheter element is introduced into the tool lumen of the imaging catheter and used to move the implant towards the implantation site. Preferably, the inner catheter member associated with the balloon also defines a lumen for receiving at least a portion of the guidewire there through to prevent the guidewire from interfering with implant deployment. An example of an inner catheter element with a balloon configured to receive a guidewire is described in U.S. Pat. No. 6,544,217.

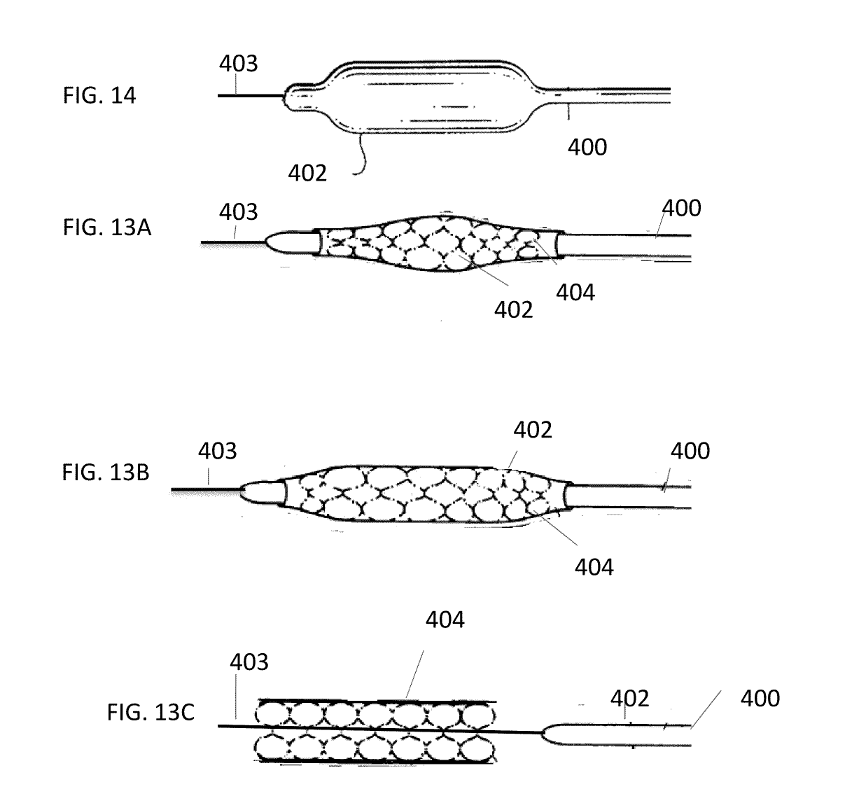

FIGS. 13A-13C depict an implant deployment mechanism that includes an expansion balloon according to one embodiment. The implant deployment mechanism includes an inner catheter element 400 that can be guided through the tool lumen or combined lumen of an imaging catheter. The inner catheter element 400 includes inflatable balloon 402. A guidewire 403, such as the imaging guidewire of the invention, extends from a lumen of the inner catheter element 400. FIG. 13A shows a stent 404 in a compressed state placed over the inflatable balloon 402. FIG. 13B shows the stent 404 in its expanded state due to the inflation of the balloon. In operation, the distal end of the imaging catheter is precisely positioned next to an implant delivery site based on images received from the imaging catheter and/or imaging guidewire. The inner catheter member 400 is distally deployed out of the lumen of the imaging catheter to position the inflatable balloon 402 and the compressed stent 404 directly within the implant delivery site. Once positioned, the inflatable balloon 402 is inflated to adjust the stent 404 from its compressed state to the expanded state. The stent 404 may be expanded to rest flush against the walls of the blood vessel. The stent 404 is configured to retain its expanded state so that the inflatable balloon 402 can be deflated and the inner catheter member 402 can be retracted (as shown in FIG. 13C). The guidewire 403 may remain disposed within the stent 404 to image the stent placement (as shown).

In an alternative embodiment, the inner catheter element with the inflatable balloon can be used to perform an angioplasty procedure. For angioplasty procedures, the inflatable balloon is introduced to a treatment site having plaque buildup. Inflation of the balloon disrupts and flattens the plaque against the vessel wall, and stretches the vessel wall, resulting in enlargement of the intraluminal passageway and increased blood flow. After such enlargement, the balloon is deflated, and the inner catheter element is removed. FIG. 14 shows the angioplasty tool for use with imaging system of the invention that includes the inner catheter element 400 and inflatable balloon 402.

Examples of transcatheter implants suitable for use with the imaging catheter of the invention include for example stents, plugs, sensors, filters and valves. The implants may include a lumen that allows the implant to ride over the guidewire. These implants are described in more detail hereinafter.

A stent is a small, typically meshed or slotted, tube-like structure made of a metal or polymer that is inserted into a blood vessel to hold the vessel open and keep it from occluding. A stent typically provides a framework for arterial lesions that are likely to embolize after angioplasty. Stents can be balloon expandable or self-expandable. Any stent configured for catheter deployment can be used, and examples of stents suitable for use with the imaging and delivery catheter of the invention are described in, for example, U.S. Pat. Nos. 5,951,586, 6,740,113, 6,387,124, and 8,133,269.

A plug is a device used to occlude a vessel to prevent fluid flow. Plugs come in a variety of shapes and sizes but are typically structured to tightly fit against the vessel wall and form a barrier within the vessel. A vascular plug can be used to temporarily occlude a blood vessel to stop blood flow during surgical treatment of the blood vessel. Alternatively, a vascular plug can permanently stop blood flow through a blood vessel that is damaged beyond repair. Plugs suitable for use in devices and methods of the invention are described in, for example, U.S. Patent Nos. 5,456,693, 6,712,836, 7,363,927, and 8,114,102.

Sensors for implantation into a vessel with the implant delivery mechanism can include sensors or monitors that detect pressure, pH, temperature, glucose, etc. A pressure sensor can be implanted into the vasculature to measure and monitor blood pressure. Pressures sensors suitable for use in devices and methods of the invention are described in, for example, U.S. Pat. No. 6,855,115. A glucose monitor measures the level of glucose in the blood and a pH monitor measures the pH of the blood. Examples of monitors suitable for use in devices and methods of the invention are described in, for example, U.S. Pat. Nos. 7,976,492, 7,881,763, and 6,689,056.

Filters may be placed into a vessel to allow fluid flow while preventing passage of undesirable particles. For example, vena cava filters are placed into the vena cava artery to provide normal blood flow while blocking passage of embolic-inducing blood clots. Filters are typically conically-shaped wire or mesh structures that are configured to anchor to a vessel's walls and span across the vessel. Filters can be balloon expendable or self-expendable. Examples of filters suitable for use in devices and methods of the invention are described in, for example, U.S. Pat. Nos. 6,099,549 and 7,534,251.

Prosthetic valves may be placed within the vasculature and are designed to replicate the function of the natural valves of the human heart. Transcatheter heart valves suitable for use in devices and methods of the invention are described in, for example, U.S. Pat. Nos. 7,981,151 and 8,070,800.

In certain aspects, the imaging catheter of the invention may be combined with an ablation tool. For example, an ablation tool can be introduced into the tool lumen 1030 or combined lumen 1055, shown in FIGS. 10 and 11, respectively. The ablation tool can be extended from the catheter lumen and into a vessel, such as a blood vessel, to perform ablation therapy. The imaging catheter and/or guidewire can be used to image the vessel before, during, and after the ablation therapy. For example, the imaging guidewire can image the ablation procedure performed along the side of the guidewire and the imaging catheter with a distal imaging element can image the procedure performed in front of the imaging catheter. There are several different types of ablation therapies. In one aspect, an ablation tool is used to remove an unwanted or damaged vein by delivering energy (RF energy, laser energy, etc) within a vein to shrink and ultimately close the vein. In another aspect, an ablation tool is used to treat heart arrhythmia disorders by ablating abnormal heart tissue to create scar tissue and disrupt the conduction pathway that lead to the disruption. In another example, the ablation tool is used to perform an atherectomy procedure to ablate arethoma or plaque within a vessel. Arethoma is an accumulation and swelling in artery walls made up of (mostly) macrophage cells, or debris, and containing lipids (cholesterol and fatty acids), calcium and a variable amount of fibrous connective tissue.

In some embodiments, the ablation tool includes at least one electrode. The electrodes can be arranged in many different patterns along the ablation tool. For example, the electrode may be located on a distal end of the ablation tool. In addition, the electrodes may have a variety of different shape and sizes. For example, the electrode can be a conductive plate, a conductive ring, conductive loop, or a conductive coil. In one embodiment, the at least one electrode includes a plurality of wire electrodes configured to extend out of the distal end of the imaging electrode.

The proximal end of the ablation tool is connected to an energy source that provides energy to the electrodes for ablation. The energy necessary to ablate cardiac tissue and create a permanent lesion can be provided from a number of different sources including radiofrequency, laser, microwave, ultrasound and forms of direct current (high energy, low energy and fulgutronization procedures). Radiofrequency (RF) has become the preferred source of energy for ablation procedures. Any source of energy is suitable for use in the ablation tool of the invention. Preferably, the source of energy chosen does not disrupt the imaging of the vessel during the procedure with the imaging guidewire and/or imaging catheter.

In operation, the imaging guidewire can be used to locate a treatment site within the vasculature that requires ablation. Once the treatment site is located, the ablation tool is deployed from the tool lumen of the imaging catheter. The electrodes located on the distal end can be placed against the treatment site and energized by an energy source operably associated with the electrodes. The energized electrodes ablate the tissue at the treatment site. In one embodiment, the imaging guidewire and imaging catheter image the luminal surface and lumen during the ablation therapy. For example, the imaging guidewire parallel to the deployed imaging tool can image the ablation during the procedure and the distal facing imaging element on the imaging guidewire can image the procedure from behind. In an alternative embodiment, the electrodes deploy several rounds of ablation therapy and the imaging catheter and imaging guidewire are used to image the ablated luminal surface between each round of energy.

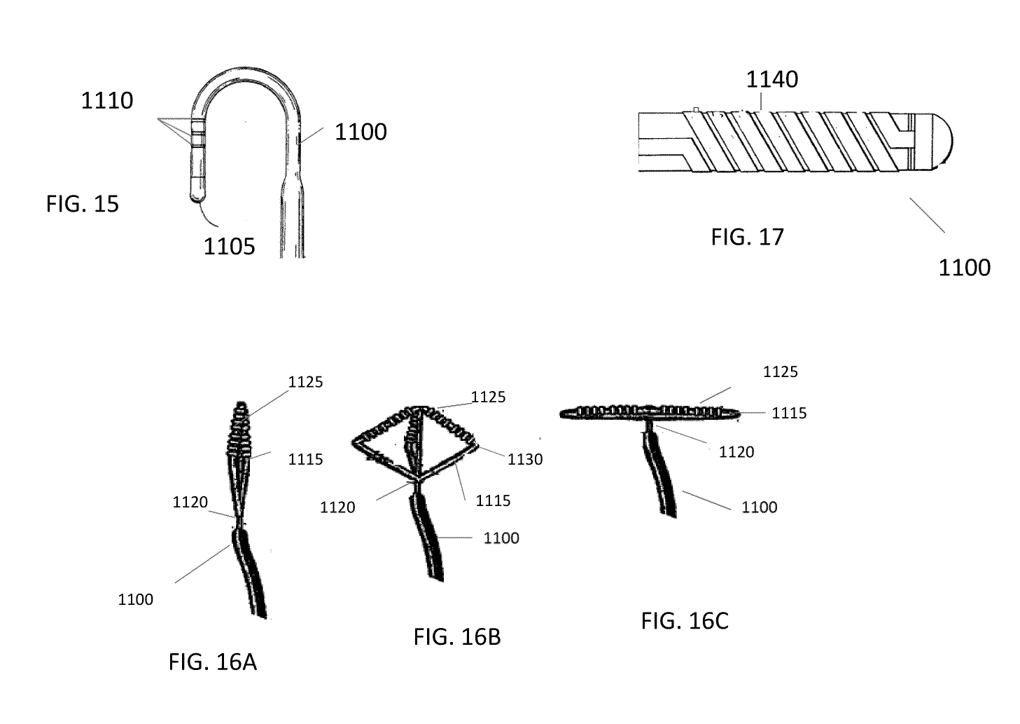

FIGS. 15-18 depicts several ablation tools suitable for use with the imaging catheter of the invention. FIG. 15 shows a distal end of an ablation tool 1100 that includes a plurality of ring electrodes 1110 and tip electrode 1105. FIG. 17 depicts a spiral electrode 1140 wrapped around the distal end of the ablation tool 1110. The distal end of the ablation tool 1110 shown in FIGS. 15 and 17 may be flexible to allow the ablation tool to press against the surface of tissue to be ablated. Examples of flexible electrode tips and methods of making flexible electrode tips that are suitable for use with the imaging catheter are described in U.S. Pat. No. 8,187,267. The entirety of which is incorporated by reference.

FIGS. 16A-16C depicts an expandable ablation tool with a distal end having a plurality of arms 1115. The arms 1115 are expandable from a center post 1120. Each arm 1115 includes a hinge 1130 and is coupled to a base ring member 1135. The ring member can be slideably moved along the center post to move the plurality of arms from the contracted position (shown in FIG. 12A), to the partially expanded position (shown in 12B) to the fully expanded position (shown in FIG. 12C). Each arm 1115 may include a wire electrode 1125 wrapped around each arm. The ablation tool, shown in FIGS. 16A-16C, is designed to expand so that the electrodes 1125 press against a vessel surface during ablation. The ablation tool shown in FIGS. 16A-16C is described in more detail in U.S. Pat. No. 7,993,333.

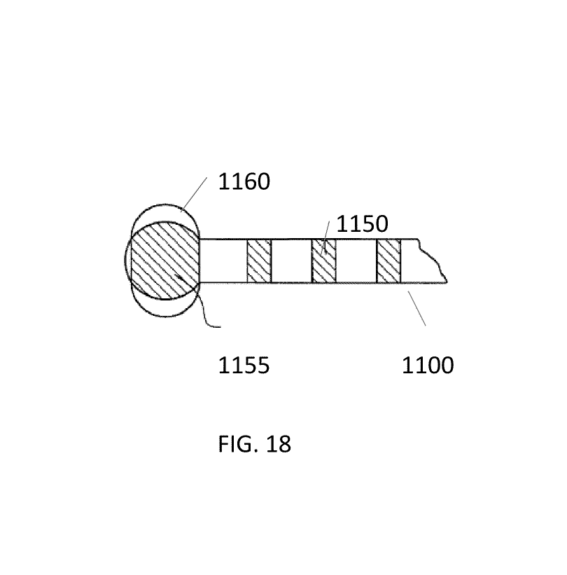

FIG. 18 depicts a balloon ablation tool that includes an inflatable balloon 1160 with balloon electrode 1155. The inflatable balloon 1160 inflates to press the electrode against a vessel surface during ablation. The balloon ablation tool includes a lumen (not shown) to introduce air or water into the balloon 1160 for inflation. Optionally and as shown, the balloon ablation tool may also include one or more ring electrodes 1150. The ablation tool shown in FIG. 18 is described in more detail in U.S. Pat. No. 6,379,352.

In other aspects, the imaging catheter of the invention may be combined with an extraction tool for use in, for example, an atherectomy procedure. Atherectomy procedures involve removing the arethoma/plaque burden within the vessel by mechanically breaking up and removing plaque from the vessel lumen to re-canalizing blocked vasculature. Increasing the vessel lumen by removing the plaque burden improves downstream wound healing, reduces claudication and pushes amputation levels more distal. While atherectomy is usually employed to treat arteries it can be used in veins and vein grafts as well. The extraction tool can be introduced into the tool lumen 1030 or combined lumen 1055, as shown in FIGS. 10 and 11 respectively. The extraction tool can be deployed from the imaging catheter into a vessel to mechanically break up and/or to remove plaque from the vessel.

In certain embodiments, the extraction tool includes a distal end that can be extended from the tool lumen of the imaging catheter. The distal end of the extraction tool includes one or more cutting elements. Typically, a proximal portion of the extraction tool is formed as part of or operably coupled to a drive shaft. The drive shaft may be coupled to a motor to provide rotational motion using any conventional means. A drive shaft suitable for use to impart rotation of the extraction tool is described in, for example, U.S. Pat. No. 5,348,017, U.S. patent publication number 2011/0306995, and co-assigned pending U.S. patent application number 2009/0018393 (as applied to rotating imaging sensors). Rotation of the drive shaft causes rotation of the distal end of the extraction tool. In operation, the distal end of the extraction is deployed from the tool lumen of the imaging catheter. Forward movement and/or rotation of the distal end of the extraction tool cause the one or more cutting element to engage with the plaque or other unwanted substances within a vessel. The cutting elements shave, morcellate, grind, or cut off plaque from the luminal surface to clear the occlusion within the vessel.

In certain embodiments, the extraction tool further defines a removal lumen extending from an opening located at the distal end of the extraction tool to an opening connected to a vacuum source. The vacuum source removes via suction plaque that has been shaved, morcellated, or cut off from the luminal surface. Alternatively, the imaging catheter may further include a removal lumen that extends from the distal end of the imaging catheter to an opening operably associated with a vacuum source. In this embodiment, morcellated or shaved plaque can be suctioned from the vessel through the removal lumen.

The cutting elements used in the present invention will usually be formed from a metal, but could also be formed from hard plastics, ceramics, or composites of two or more materials, which can be honed or otherwise formed into the desired cutting edge. In certain embodiments, the cutting blades are formed as coaxial tubular blades with the cutting edges defined in aligned apertures therein. It will be appreciated that the present invention is not limited to any particular cutting element, and the cutting element may include a variety of other designs, such as the use of wiper blades, scissor blades or the like. The cutting elements can have razer-sharp smooth blade edges or serrated blade edges. Optionally, the cutting edge of either or both the blades may be hardened, e.g., by application of a coating. A preferred coating material is titanium nitride.

In operation, the imaging catheter can be used to locate a treatment site within the vasculature that requires extraction of plaque, damaged or malignant tissue, or any other unwanted substance within the vasculature. Once the treatment site is located, the extraction tool is deployed from the tool lumen of the imaging catheter. The cutting elements of the distal end are placed next to and/or against the treatment site. The cutting elements are then translated longitudinally within the vessel (i.e. forward and backward movement) and/or rotated. The translation and/or rotation of the cutting elements against the treatment site allow the cutting elements to morcellate or shave off the plaque. In one embodiment, morcellated or shaved plaque can be disposed of through a removal lumen via vacuum pressure. In certain embodiments, the plaque is morcellate and removed from the vessel in piecemeal fashion and the imaging catheter is used to image the vessel between each round of plaque removal.

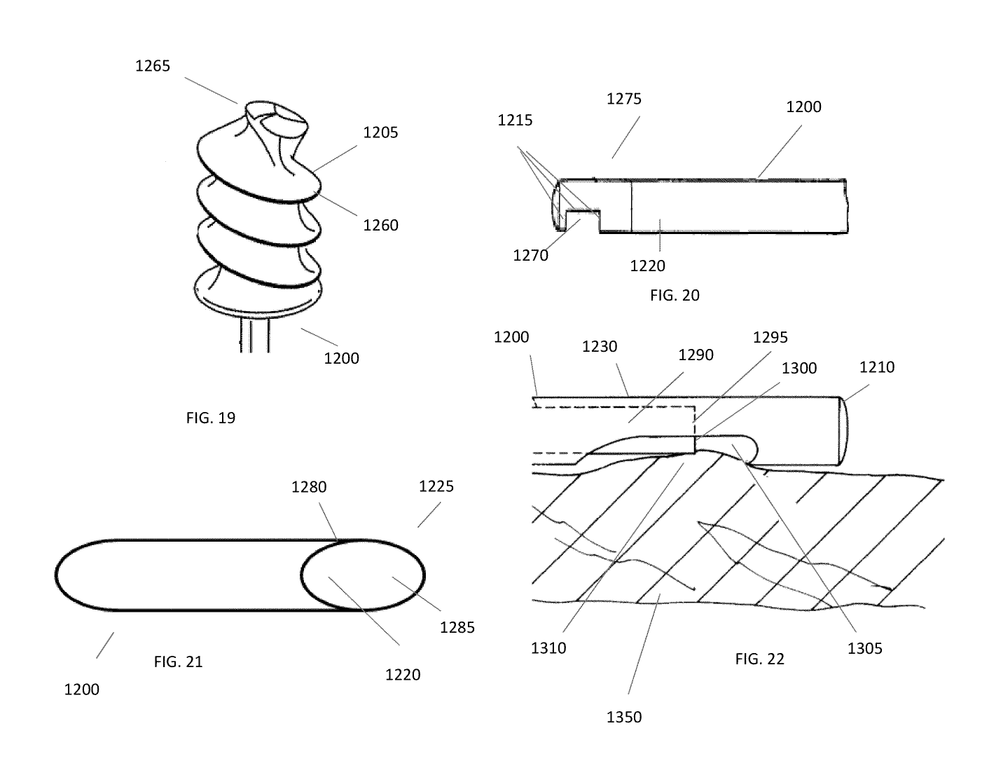

FIGS. 19-23C depict various embodiments of a distal end of the extraction tool suitable for use with the imaging catheter of the invention.

As shown in FIG. 19, the distal end 1200 of the extraction tool includes a helical cutting element 1205. The helical cutting element 1205 has a spiral-fluted shape. The edges 1260 of the spiral are sharp blades. When rotated, the helical cutting element 1205 grounds plaque within the vessel. The tip 1265 of the helical cutting element 1205 can be formed as a bladed point. The bladed point tip will assist in morcellating plaque that may be present in front of the extraction tool.

FIG. 20 depicts a distal end 1200 of an extraction tool according to one embodiment. The distal end 1200 of the extraction tool includes a recessed cutting element 1275. The recessed cutting element 1275 includes a recess 1260 within the distal end 1200 formed by edges 1260. One or more of the edges 1260 that form the recess 1260 constitute cutting blades. Optionally and as shown, the extraction tool includes a removal lumen 1220 and the recess 1260 provides access to the removal lumen 1220. The removal lumen 1220 can extend along the length of the extraction tool and operably couple to a vacuum source. In operation, the recessed cutting element 1275 is distally deployed from the tool lumen of the imaging catheter. The recessed cutting element 1275 can be moved forward and backwards and rotated to shave off or morcellate any plaque or unwanted substance that is placed within the recess 1260 via the blade edges 1260. The shaved off or morcellated plaque can be removed from the vessel through the removal lumen 1220.

FIG. 21 depicts a distal end 1200 an extraction tool according to another embodiment. The extraction tool includes a tubular member with a bladed end 1225 at the distal end 1220. The bladed end 1225 is formed by a sharp edge 1280. The bladed end 1225 can be open or closed. As shown in FIG. 21, the bladed end is open and includes opening 1285. The opening 1285 leads to a removal lumen 1220. In order to morcellate plaque and other unwanted substances, the distal end 1200 of extraction tool is deployed from the tool lumen of the imaging catheter. As the distal end 1200 is moved forward and rotated, the sharp edge 1280 cuts through and morcellates plaque present in front of the distal end 1200. The shaved off or morcellated plaque can be removed from the vessel through the removal lumen 1220.

FIG. 22 depicts the distal end 1200 of an extraction tool according to yet another embodiment. The extraction tool includes an outer tubular member 1210 that defines a removal lumen 1230 and an inner tubular member 1290 disposed within the removal lumen 1230. The outer tubular member 1210 includes a window 1305. The removal lumen 1230 can be operably coupled to a vacuum source. The inner tubular member 1290 can be moved forward and backward and rotated with respect to the outer tubular member 1210. The inner tubular member includes the same elements as the extraction tool shown in FIG. 21. The inner tubular member 1290 includes a bladed end 1295. The bladed end 1295 can be open or closed. The bladed end 1295 is formed by a sharp edge 1300. In operation, the distal end 1200 of the extraction tool is deployed from the tool lumen of the imaging catheter. The window 1305 of the outer tubular member 1210 is placed against plaque 1310 protruding from the vessel wall 1350. The inner tubular member 1290 can be moved forward and backwards and rotated within outer tubular member to morcellate and shave off any plaque placed within the window 1305. Removed plaque can be suctioned out of the vessel through the removal lumen 1230.

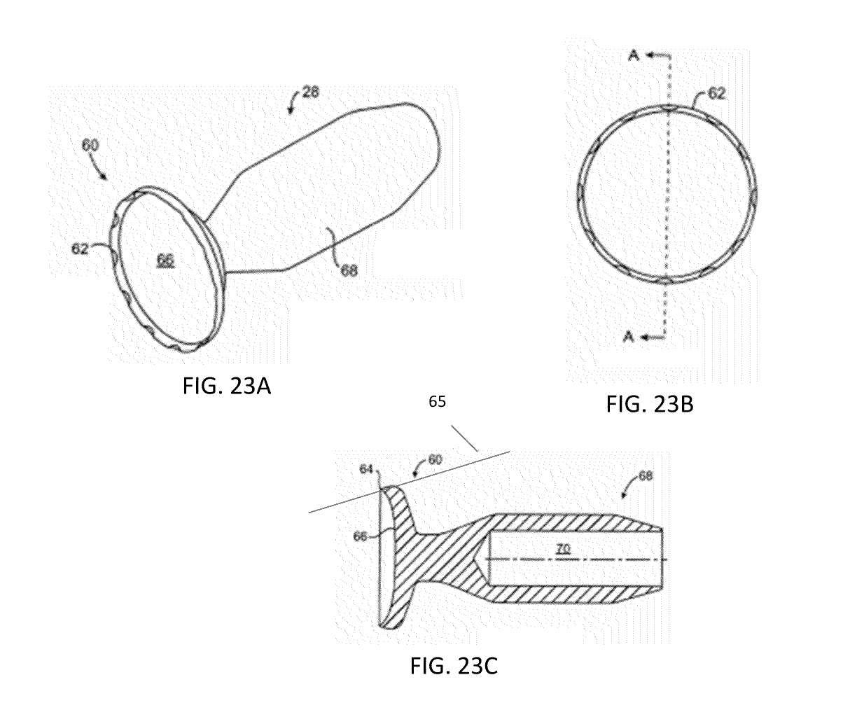

FIGS. 23A through 23C show some exemplary embodiments of a distal end 60 of an extraction tool 28. The distal portion 60 of the extraction tool 28 can include a serrated knife edge 62 or a smooth knife edge 64 and a curved or scooped distal surface 66. The distal portion 60 may have any suitable diameter or height. In some embodiments, for example, the diameter across the distal portion 60 may be between about 0.1 cm and about 0.2 cm. A proximal portion 68 of the cutter 28 can include a channel 70 that can be coupled to the drive shaft 36 that rotates the cutter. In any of the foregoing embodiments, it may be advantageous to construct a serrated knife edge 62, a smooth knife edge 64, or a scooped distal surface 66 out of tungsten carbide, stainless steel, titanium or any other suitable material.

As shown in FIG. 23C, the cutter 28 has a beveled edge 64, made of tungsten carbide, stainless steel, titanium or any other suitable material. The beveled edge 64 is angled inward, toward the axis of rotation (or center) of the cutter 28, creating a “negative angle of attack” 65 for the cutter 28. Such a negative angle of attack may be advantageous in many settings, when one or more layers of material are desired to be removed from a body lumen without damaging underlying layers of tissue. Occlusive material to be removed from a vessel typically has low compliance and the media of the vessel (ideally to be preserved) has higher compliance. A cutter 28 having a negative angle of attack may be employed to efficiently cut through material of low compliance, while not cutting through media of high compliance, by allowing the high-compliance to stretch over the beveled surface of cutter 28.

In yet another embodiment, an extraction tool can include an inner catheter element with an inflatable cutting balloon. This embodiment is substantially similar to the angioplasty tool, as shown in FIG. 14, except that the balloon further includes one or more cutting elements that are configured to remove tissue from the luminal surface when the inflatable balloon is engaged with the luminal surface. The inflatable cutting balloon includes one or more blade elements on the outside of the balloon. The inner catheter element may be operably associated with a drive shaft coupled to a motor. Rotation of the drive shaft, as driven by the motor, may cause rotation of the inflatable balloon. As an inflated balloon rotates against the luminal surface, the cutting elements shave off any tissue located on the luminal surface.

In addition, the devices and methods of the invention may also involve the introduction of an introducer sheath. Introducer sheaths are known in the art. Introducer sheaths are advanced over the guidewire into the vessel. A catheter or other device may then be advanced through a lumen of the introducer sheath and over the guidewire into a position for performing a medical procedure. Thus, the introducer sheath may facilitate introducing the catheter into the vessel, while minimizing trauma to the vessel wall and/or minimizing blood loss during a procedure.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1) An imaging and extraction system comprising
an elongate member comprising at least one first imaging element, wherein the first imaging element includes a first acoustic-to-optical transducer; and
a catheter defining a lumen and comprising at least one second imaging element and a cutting element, wherein the catheter is configured to receive at least a portion of the elongate member within the lumen and is configured to move along a length of the elongate member, and the second imaging element includes a second acoustic-to-optical transducer.

2) The system of claim 1, wherein the elongate member is a guidewire.

3) The system of claim 1, wherein the first imaging element includes at least one optical fiber.

4) The system of claim 1, wherein the elongate member further comprises a pressure sensor.

5) The system of claim 1, wherein the second imaging element includes at least one optical fiber.

6) The system of claim 1, wherein the first acoustic-to-optical transducer and the second acoustic-to-optical transducer are the same.

7) The system of claim 6, wherein the first and second acoustic-to-optical transducers include a Bragg grating element.

8) The system of claim 1, wherein the cutting element is configured to remove an atheroma substance from an imaging surface.

9) The system of claim 9, wherein the cutting element is operably associated with a drive shaft.

10) The system of claim 9, wherein the drive shaft is configured to rotate the cutting element to morcellate or shave off plaque from the imaging surface.

11) The system of claim 1, wherein the cutting element is operably associated with a balloon.

12) The system of claim 8, wherein the catheter further includes a disposal member that is configured to dispose of the substance as removed from the imaging surface.

13) The system of claim 1, wherein the first imaging element and the second imaging element include at least one other transducer.

14) The system of claim 13, wherein the at least one other transducer is an electrical-to-acoustic transducer or an optical-to-acoustic transducer.

15) The system of claim 13, wherein the at least one other transducer includes a piezoelectric element.

16) A method for intraluminal treatment, the method including the steps of
introducing an elongate member into a lumen, wherein the elongate member comprises at least one first imaging element and the first imaging element a first acoustic-to-optical transducer;
imaging a surface of the lumen with the elongate member to locate atheroma material;
guiding a catheter over at least a portion of the elongate member towards the atheroma material, wherein the catheter comprises at least one second imaging element and a cutting element, and the second imaging element includes a second acoustic-to-optical transducer;
imaging the surface of the lumen, as the catheter is guided towards the plaque, to position the catheter for atheroma material removal; and
removing the atheroma material from the surface with the cutting element.

17) The method of claim 16, further comprising the steps of
imaging the step of removing with the elongate member, catheter, or both.

18) The method of claim 16, wherein the cutting element is operably associated with a drive shaft.

19) The method of claim 18, wherein the step of removing includes rotating the cutting element to morcellate or shave off the atheroma material.

20) The method of claim 16, wherein the cutting element is also operably associated with a balloon.

21) The method of claim 16, further comprising the step of disposing the removed atheroma material from the lumen.

22) The method of claim 16, wherein the first and second acoustic-to-optical transducers are the same and include a Bragg grating element.

23) The method of claim 16, wherein the imaging element and the second imaging portion include at least one other transducer.

24) The method of claim 23, wherein the at least one other transducer is an electrical-to-acoustic transducer or an optical-to-acoustic transducer.

25) The method of claim 23, wherein the at least one other transducer is a piezoelectric element or a photoacoustic element.
